(19) United States
(12) Patent Application Publication
(10) Pub. No.: US 2023/0098327 A1
(43) Pub. Date: Mar. 30, 2023

(54) SLEEP-INDUCING BEVERAGE COMPOSITION, ITS METHOD OF PRODUCTION AND THE METHOD OF USE

(71) Applicant: Premier Nutrition Company, LLC, Emeryville, CA (US)

(72) Inventors: Madhavi Ummadi, Emeryville, CA (US); Nishtha Lukhmana, Emeryville, CA (US); Venkata Sarath Pamu, Emeryville, CA (US); Liqiang Zhao, Emeryville, CA (US); Kimberly Ann Swisher Lutze, Emeryville, CA (US); Emily Elizabeth Hurt, Emeryville, CA (US)

(21) Appl. No.: 17/899,150

(22) Filed: Aug. 30, 2022

Related U.S. Application Data
(60) Provisional application No. 63/238,392, filed on Aug. 30, 2021.

(51) Int. Cl. A61K 35/20; A61K 9/00; A61K 47/42; A61K 47/38; A61K 33/30; A61K 33/06; A61K 31/07; A61K 31/122; A61K 31/355; A61K 31/593; A61K 31/4415; A61K 31/375; A61K 31/519; A61K 31/14; A61K 47/36; A61K 47/24; A61K 38/08

(52) U.S. Cl. CPC A61K 35/20; A61K 9/0095; A61K 47/42; A61K 47/38; A61K 33/30; A61K 33/06; A61K 31/07; A61K 31/122; A61K 31/355; A61K 31/593; A61K 31/4415; A61K 31/375; A61K 31/519; A61K 31/14; A61K 47/36; A61K 47/24; A61K 38/08

(57) ABSTRACT

Described herein is an innovative composition that improves sleep quality. This composition includes two important systems: 1) a sleep-inducing system that is being used for the first time in food and beverage products; and 2) a unique stabilizer system that provides a robust rheology and greater stability than other stabilizers. The two systems can be incorporated, individually or in combination, into food and beverage products, including but not limited to protein shakes and powders. Together, they enable this composition to aid in relaxation and optimal sleep quality, better sensory taste experiences than current market products, longer shelf life and robust stability. This disclosure also addresses a relevant need in the art for a beverage composition that promotes calmness and reduces stress by using natural ingredients, more specifically made from milk casein. In summary, the sleep-inducing RTD (ready-to-drink) and RTM (ready-to-mix) compositions described herein include a stabilizer composition that improves shelf-life stability and contributes to enhanced specific sensory attributes without any adverse effects.

### FIELD OF THE DISCLOSURE

The present disclosure introduces an innovative composition that improves sleep quality. The composition created is unique and includes: 1) a relaxation and/or sleep-inducing composition that is a unique composition being used for the first time in food and beverage products; and 2) a unique stabilizer composition that provides better rheology and greater stabilization than other stabilizers in food and beverage compositions. In addition, the unique sleep-inducing composition can be created following unique processing methods to create a sleep-inducing composition to aide in relaxation that has increased shelf stability attributes. The unique sleep-inducing composition and stabilizing composition can be incorporated alone or in combination into consumer food and beverage products, including protein shakes and powders. Together, they enable this composition to aid in relaxation, induce sleep and provide longer shelf life and better sensory taste experiences than current market products.

### BACKGROUND

Sleep deprivation could be a consequence of multiple health problems or a cause of many major health risks and is a significant public health concern. The complex relationship between wellbeing, health and productivity, and sleep impairment can act as a moderator and a disturbed sleep quality can negatively impact the mental and physical wellbeing of a person. Furthermore, insomnia is a broad term used to characterize a person's difficulty in inducing or maintaining sleep. Insomnia can be found in 30% or more of the population each year, causing disruption in many people's lives. The severity of insomnia varies depending on personal circumstances that include but are not limited to lifestyle, food habits, exercise, medical history, etc. but even minimal insomnia can have lasting effects on a person. There are many compositions on the market that are designed to help with sleep quality, sleep duration and the ease of falling asleep, however these products can be difficult for a consumer to take, when in a pill or other formats and in certain instances may have side effects or can present long term addiction. Also, beverage compositions that include protein shakes and protein powders to create protein shake that further include functional ingredients on the market have long suffered from poor sensory attributes and shelf stability, which translate into a product not desired by consumers. Specifically, from a rheological characteristic (and the resultant desirability of the consumer end product) high elastic modulae and high viscosity modulae create an unsatisfactory mouthfeel for consumers and a reduced shelf stability time period before the product has an unsatisfactory texture that can include solids precipitating out in the shake. Although many stabilizers have been used to solve these problems, they can sometimes alter the beverage's taste, sensory attributes, or prevent the addition of micronutrients. There is thus a need in the art for a beverage composition that promotes calmness, reduces stress and increase relaxation by using a combination of ingredients that can include natural ingredients and naturally occurring ingredients. The sleep-inducing RTD (ready-to-drink) and RTM (ready-to-mix) compositions further include a stabilizer composition that improves shelf-life stability, made following a unique processing method, and contributes to enhancing specific sensory attributes without any adverse effects.

### SUMMARY OF THE DISCLOSURE

Disclosed herein is a sleep-inducing RTD (ready-to-drink) and RTM (ready-to-mix) compositions that include functional ingredients in particular a relaxation and/or sleep-inducing composition and a beverage stabilizer composition comprising a stabilizing agent, a chelator, and an emulsifier. The beverage composition created has been shown through research and scientific substantiation to promote calmness and optimal sleep and to create a shelf stable product with sensory characteristics that consumers desire. The beverage composition includes a stabilizing agent that may be selected from the group consisting of at least one cellulose gel/gum, a starch, a gellan gum, and any combination thereof. In some embodiments, the cellulose gel/gum can include carboxy methylcellulose, microcrystalline cellulose, and any combination thereof. In other embodiments, the starch may be a waxy potato starch. In still other embodiments, the gellan gum may be a high acyl gellan gum, a low acyl gellan gum, and any combination thereof. In some examples, the chelator is potassium citrate. In other examples, the emulsifier is sunflower lecithin, acacia gum, monoglycerides, diglycerides and/or any combinations thereof.

Also disclosed herein is a beverage composition that is a protein shake comprising the stabilizer composition above, as well as a protein composition combined with the functional ingredients, and at least one food additive, that together creates a desired sleep-inducing beverage. In some embodiments, the protein may include a dairy protein or a plant protein. In some examples, the protein may be a milk protein isolate, a milk protein concentrate, a milk protein hydrolysate, plant proteins including but not limited to soy proteins, pea proteins and/or any combination of dairy and plant proteins. The sleep-inducing beverage may include an amount of casein hydrolysate, zinc, calcium, phosphorus, magnesium, vitamin A, vitamin K, vitamin E, vitamin D, vitamin B6, vitamin C, folate, choline and combinations thereof. The sleep-inducing beverage composition could be a ready-to-drink (RTD) or ready-to-mix (RTM) powder that when combined with a liquid ingredient creates a beverage composition. The sleep-inducing protein ready-to-drink (RTD) shake or ready-to-mix (RTM) powder composition may also include at least one additional food additive, a flavoring agent, a coloring agent, a pH adjusting agent, an antifoaming agent, sweeteners, salt, water, micronutrients or functional ingredients, fiber, and any combination thereof to create the desired beverage or pre-beverage powder composition. The additional food additive ingredient or ingredients will be dependent on the desired end product produced.

### BRIEF DESCRIPTION OF THE FIGURES

FIG. 1 is a graphical representation showing the results of a frequency sweep analysis of various shakes, including one embodiment of the protein shake described herein. The x-axis the angular frequency ω (rad/s) and the y-axis provides the loss modulus G″ (Pa) and storage modulus G′ (Pa).

FIG. 2 is a graphical representation showing the results of a strain sweep analysis of various beverages, including one embodiment of the protein shake described herein. The x-axis provides the oscillation strain γ (%) and the y-axis provides the storage modulus G′ (Pa).

FIG. 3 is a flow chart to outlines current methods of starch modifications for use in starch ingredients used in embodiments of the protein shakes described herein. (reference to http://www.intechopen.comibooksichemical-properties-of-starch/physical-and-chemical-modifications-in-starch-structure-and-reactivity)

FIG. 4 Overview of the compositions of RTD (ready-to-drink) and RTM (ready-to-make) protein shake.

FIG. 5. Is a flow chart for Tetra Therm Aseptic VTIS based on tubular heat exchanger and steam injection.

FIG. 6 is a bar graph showing comparison results of consumer preference/liking for flavor, texture and overall product when comparing current market products to embodiments of the protein shake described herein.

### DETAILED DESCRIPTION

Various embodiments of the disclosure are discussed in detail below. While specific implementations are discussed, the following information is provided for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

Several definitions that apply throughout this disclosure will now be presented. Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

As used herein, the terms “comprising,” “having,” and “including” are used in their open, non-limiting sense. The terms “a,” “an,” and “the” are understood to encompass the plural as well as the singular. Thus, the term “a mixture thereof” also relates to “mixtures thereof.”

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

As used herein, “shelf-life” refers to the time period extending from producing and packaging a beverage until the rheological profile of the beverage leads to solids precipitating in the beverage or the mouthfeel of the beverage deteriorates. It does not refer to the time period ending with the beverage being unsuitable for consumption. Shelf life of the beverages herein means a product, a protein shake, that does not have (exhibit) defects in the physical stability of the beverage composition that can include phase separation, sedimentation, creaming, or aggregation at or over a given time period.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

### I. Stabilizer Composition

Disclosed herein is a stabilizer composition, and the resultant food and beverage composition, that is useful for improving the shelf life, texture and improving the mouthfeel of RTD (ready-to-drink) and RTM (ready-to-mix) compositions, these include protein shakes comprising dairy proteins and/or plant proteins and/or a combination thereof. The novel protein shake composition includes advantages such as improved shelf-life, texture and mouthfeel imparted by the unique stabilizer composition and is demonstrated by the stable rheological properties of the beverage, including elasticity and viscosity that are an improvement and better than those found in known RTD and RTM compositions. In some embodiments, the stabilizer composition may be used in a RTD (ready-to-drink) liquid beverage and RTM (ready-to-mix) composition, such as a low acid and high acid shakes, or in a semisolid food composition, such as a yogurt or ice cream or in filled and processed meat and meat analog products, such as sausages. In some embodiments, the stabilizer composition may include at least one stabilizing agent, a chelator, and an emulsifier. In various embodiments, the stabilizer composition may further include an oligosaccharide. In still other embodiments, the stabilizer may further include milk fat globule membrane (MFGM). The stabilizer composition may have a concentration in the beverage that could vary from about 0.001% to about 0.05%. In other embodiments the stabilizer composition may have a concentration of at least 10 ppm.

The stabilizer composition of the present disclosure includes at least one stabilizing agent. As used herein, a stabilizing agent refers to any composition that improves the stability of a beverage, such as a RTD beverage, and contributes to a robust system with the desired shelf life of 12 to 14 months. The increased shelf-life stability helps ensure a RTD beverage composition has the mouth feel characteristics desired by a consumer even after it has been transported and set on a self for an extended time. In some embodiments, the stabilizing agent may include a carboxy methylcellulose gel/gum, a gellan gum (including but not limited to high acyl and low acyl gellan gum), a starch, maltodextrin, or any combination thereof. In some aspects, the cellulose gel/gum may include carboxymethyl cellulose (CMC), microcrystalline cellulose (MCC), or any combination thereof. In one non-limiting example, the cellulose gel/gum may be Avicel®. In another non-limiting example, the cellulose gel/gum may be MSM28 from the Hydrosol Company. The cellulose gel/gum may have a concentration in the beverage of about 0.01% to about 1.25%. In some examples, the cellulose gel/gum may have a concentration in the beverage of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2%, or about 1.25%. In some further examples, the cellulose gel/gum may have a concentration in the beverage of about 0.01% to about 0.02%, about 0.02% to about 0.03%, about 0.03% to about 0.04%, about 0.04% to about 0.05%, about 0.05% to about 0.06%, about 0.06% to about 0.07%, about 0.07% to about 0.08%, about 0.08% to about 0.09%, about 0.09% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, about 0.8% to about 0.9%, about 0.9% to about 1.0%, about 1.0% to about 1.1%, or about 1.1% to about 1.2%. In one non-limiting example, the cellulose gel/gum has a concentration in the beverage of about 0.1%.

In some embodiments, the stabilizing agent may include a gellan gum. The gellan gum may be high acyl gellan gum, low acyl gellan gum, or any combination thereof. In non-limiting examples of gellan gum examples include HM Pure or Kelcogel F.

In some embodiments, the stabilizing agent includes high acyl gellan gum. The high acyl gellan gum may have a concentration in the beverage of between about 25 ppm to about 600 ppm. In some examples, the high acyl gellan gum may have a concentration of about 25 ppm, 50 ppm, 75 ppm, 100 ppm, 150 ppm, 200 ppm, 250 ppm, 300 ppm, 350 ppm, 400 ppm, 450 ppm, 500 ppm, 550 ppm, or about 600 ppm. In further examples, the high acyl gellan gum may have a concentration of about 25 ppm to about 50 ppm, about 50 ppm to about 75 ppm, about 75 ppm to about 100 ppm, about 100 ppm to about 150 ppm, about 150 ppm to about 200 ppm, about 200 ppm to about 250 ppm, about 250 ppm to about 300 ppm, about 300 ppm to about 350 ppm, about 350 ppm to about 400 ppm, about 400 ppm to about 450 ppm, about 450 ppm to about 500 ppm, about 500 ppm to about 550 ppm, about 550 ppm to about 600 ppm. In one non-limiting example, the high acyl gellan gum has a concentration of about 500 ppm.

In some embodiments the stabilizing agent may include low acyl gellan gum. The low acyl gellan gum may have a concentration in the beverage of about 25 ppm to about 250 ppm. In some examples, the low acyl gellan gum has a concentration of about 25 ppm, 50 ppm, 75 ppm, 100 ppm, 125 ppm, 150 ppm, 175 ppm, 200 ppm, 225 ppm, or about 250 ppm. In further examples, low acyl gellan gum may have a concentration of about 25 ppm to about 50 ppm, about 50 ppm to about 75 ppm, about 75 ppm to about 100 ppm, about 100 ppm to about 125 ppm, about 125 ppm to about 150 ppm, about 150 ppm to about 175 ppm, about 175 ppm to about 200 ppm, 200 ppm to about 225 ppm, or about 225 ppm to about 250 ppm. In one non-limiting example, the low acyl gellan gum has a concentration in the beverage of about 25 ppm.

In some embodiments, the stabilizing agent may include both high acyl gellan gum and low acyl gellan gum. In some aspects, the ratio of the concentration of high acyl gellan gum to the concentration of low acyl gellan gum may be between about 1:1 and about 4:1.

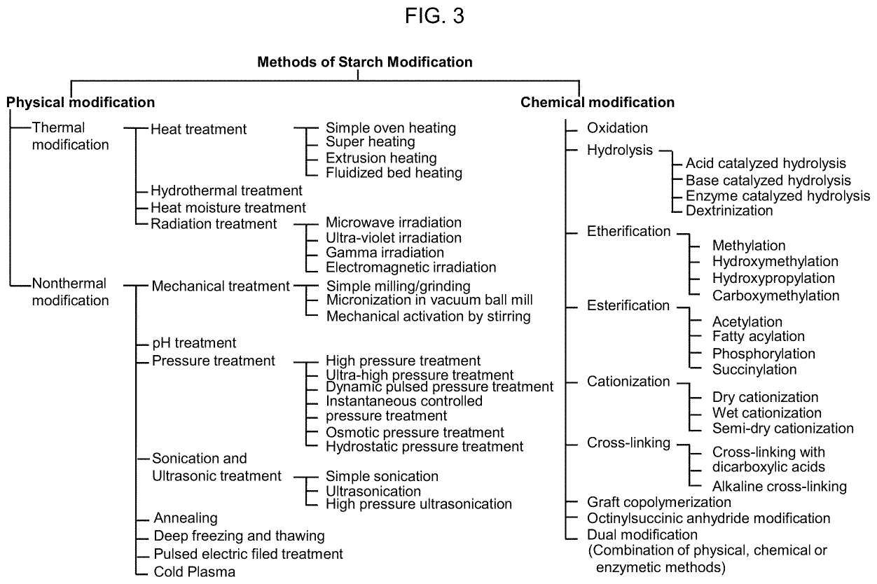

In some embodiments, the stabilizing agent may include a starch. In some embodiments, the starch may be waxy and non-waxy starches from crops as potato, wheat, cassava, corn, or other starches recognized by those skilled in the art. In some aspects, the starch may have a chemical or physical modification to improve its stabilizing qualities. FIG. 3 provides an overview of types of modifications that are known in the industry and can be used to improve stabilizing qualities of a starch. In some aspects, the starch may have a concentration in the beverage of about 0.3% to about 3%.

In some embodiments, the stabilizer composition includes a chelator and/or buffer. The chelator may include potassium citrate, calcium citrate, sodium citrate, or magnesium citrate, glycine, citric acid, gluconic acid, tartaric acid, hexametaphosphoric acid, pyrophosphoric acid, tripolyphosphoric acid, phytic acid, ethylenediaminetetraacetic acid (EDTA), calcium disodium EDTA, disodium EDTA, and other chelating agents known to those skilled in the art. In one example, the chelator is potassium citrate. The chelator may have a concentration in the beverage between about 0.01% and about 0.6%. In some examples, the chelator may have a concentration of about 0.01%, 0.02%, 0.03%, 0.04%, 0.05%, 0.06%, 0.07%, 0.08%, 0.09%, 0.1%, 0.2%, 0.3%, 0.4%, 0.5%, or 0.6%. In further examples, the chelator may have a concentration from about 0.01% to about 0.02%, about 0.02% to about 0.03%, about 0.03% to about 0.04%, about 0.04% to about 0.05%, about 0.05% to about 0.06%, about 0.06% to about 0.07%, about 0.07% to about 0.08%, about 0.08% to about 0.09%, about 0.09% to about 0.1%, about 0.1% to about 0.2%, about 0.2% to about 0.3%, about 0.3% to about 0.4%, about 0.4% to about 0.5%, or about 0.5% to about 0.6%. In one non-limiting example, the chelator is potassium citrate, which has a concentration in the beverage of about 0.05%.

In some embodiments, the stabilizer composition may include an emulsifier. Those having skill in the art will recognize that emulsifiers have a hydrophilic/lipophilic balance ratio (HLB) that ranges from 0 to 20. In some embodiments, the emulsifier may have a HLB that ranges from about 5 to about 10. For example, the HLB of the emulsifier may be about 5, 6, 7, 8, 9, or about 10. In some non-limiting examples, the HLB of the emulsifier is about 6 to about 8.

In some embodiments, the stabilizer may further include at least one additional ingredient may include an oligosaccharide. In some embodiments, the oligosaccharide may include galactooligosaccharides (GOS), fructooligosaccharides (FOS), or other oligosaccharides known by those skilled in the art. In one example, the oligosaccharide is GOS. Without being bound to any particular theory, GOS may improve shelf life and mouthfeel by binding to protein moieties or other polymers in the beverage. In some embodiments, the concentration of oligosaccharides in the beverage may be from about 0.05% to about 4%.

In some embodiments, the stabilizer may further include at least one additional ingredient such as a milk fat globule membrane (MFGM). The MFGM can be a synthetic MFGM or natural MFGM and derived from a variety of animals. The animals the MFGM are derived can include but are not limited to cows or goats. In some examples, the MFGM or buttermilk powder may be Lipid 100® from Fonterra. Without being bound to any particular theory, MFGM may increase shelf life and mouthfeel due to its lipid profile. In some examples, the MFGM has a concentration in the beverage of about 0.1% to about 4%.

In some embodiments, the protein shake may contain a source of fiber. The fiber source can be an amount that is considered a high source of fiber based on a persons recommended daily average source. Products considered “high”, “rich in” or “excellent source of” fiber is a food or beverage composition that contains 20% or more of the daily value (DV) per refence amount customarily consumed (RACC). In non-limiting examples, the fiber source may include inulin, fructo-oligosccharides, resistant maltodextrins, galacto-oligosaccharides, gum arabic, partially hydrolyzed guar gum (PHGG), soluble corn fiber, or any other fiber sources known to those skilled in the art. In some aspects, the fiber may have a concentration from about 2.4% to about 7.5%. In some examples, the fiber may have a concentration of 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9, 4.0%, 4.1%, 4.2% or about 4.3%. In some further examples, the fiber may have a concentration from about 2.5% to about 3.0%, about 3.0% to about 3.5%, about 3.5% to about 4.0%, about 4.0% to about 4.5%, about 4.5% to about 5.0%, about 5.0% to about 5.5%, about 5.5% to about 6.0%, about 6.0% to about 6.5%, about 6.5% to about 7.0%, or about 7.0% to about 7.5%. In one non-limiting example, the fiber may have a concentration of about 3.5% in the beverage formulation. In one non-limiting example the beverage may contain 5.6 g or more of fiber per 240 ml. Further the amount of fiber may be 5.6 g per 8 oz/240 ml of the beverage. In an additional embodiment the beverage may be 6.125 g per 8.75 oz. Dependent on the fluid size of the beverage product in a non-limiting example the amount of fiber will be 0.7 g per oz of the beverage in order produce a high fiber beverage composition.

In some embodiments, the emulsifier may be a lecithin, steroyl lactylates, guar gum, monoglycerides, diglycerides, polysorbates, propylene glycol esters, sucrose esters, polyglycerol esters, polyglycerol polyricinoleate, ammonium phophatide, acacia gum and other emulsifiers known by those skilled in the art. In non-limiting examples the emulsifiers can include Soya Lecithen Granules G, Soya Lecithin Powder P, Soya Lecithin Liquid, distilled glycerin monostearate, potassium stearate, calcium stearoyl lactylate (CLS), DATEM, glyceryl monostearate, mono propylene glycol, SPAN 80, sodium stearoyl lactylate (SSL), Tween (polysorbate 80), sodium stearate, glycerol triacetate, sugar esters, polyglycerol esters of fatty acid, calcium stearate, polyglycerol polyricinoleate (PGPR) and combinations of the above. In some aspects, the emulsifier is a lecithin, such as sunflower lecithin, soy lecithin, egg lecithin, canola lecithin, rapeseed lecithin, corn lecithin, or other lecithins known in the art. In some aspects, the emulsifier may have a concentration from about 0.03% to about 0.8%. In some examples, the emulsifier may have a concentration in the beverage of about 0.03%, 0.04%, 0.05%, 0.06%, 0.07%, 0.08%, 0.09%, 0.1%, 0.2%, 0.3%, 0.4%, 0.5%, 0.6%, 0.7%, or about 0.8%. In some further examples, the emulsifier may have a concentration from about 0.03% to about 0.04%, about 0.04% to about 0.05%, about 0.05% to about 0.06%, about 0.06% to about 0.07%, about 0.07% to about 0.08%, about 0.08% to about 0.09%, about 0.09% to about 0.1%, about 0.1% to about 0.2%, about 0.2% to about 0.3%, about 0.3% to about 0.4%, about 0.4% to about 0.5%, about 0.5% to about 0.6%, about 0.6% to about 0.7%, or about 0.7% to about 0.8%. In one non-limiting example, the emulsifier has a concentration in the beverage of 0.03%.

In some embodiments, the protein shake may further include at least one additional ingredient that may comprise an oil. The oil may have a concentration in the beverage between about 0.1% and about 5%. In some embodiments, the oil may include medium-chain triglyceride (MCT) oil, canola oil, corn oil, soy oil, high oleic sunflower oil (HOSO), soybean oil, pumpkin seed oil, grapeseed oil, peanut oil, sesame oil, flaxseed oil, sunflower seed oil, vegetable oil, or any combinations thereof. For example, the oil may have a concentration of about 0.1%, 0.2%, 0.3%, 0.4%, 0.5%, 0.6%, 0.7%, 0.8%, 0.9%, 1.0%, 1.5%, 2.0%, 2.5%, 3.0%, or about 3.5%. In other examples, the chelator may have a concentration of about 0.1% to about 0.2%, about 0.2% to about 0.3%, about 0.3% to about 0.4%, about 0.4% to about 0.5%, about 0.5% to about 0.6%, about 0.6% to about 0.7%, about 0.7% to about 0.8%, about 0.8% to about 0.9%, about 0.9% to about 1.0%, about 1.0% to about 1.5%, about 1.5% to about 2.0%, about 2.0% to about 2.5%, about 2.5% to about 3.0%, or about 3.0% to about 3.5%. In one non-limiting example, the oil has a concentration of about 0.3% in the beverage.

The stabilizer composition may be used in a variety of food and beverage products. In some embodiments, the stabilizer composition may be used in a beverage composition, such as a shake, a juice, or other liquid food products. In additional embodiments, the stabilizer composition may be used in a semiliquid food composition, such as yogurt, ice cream, custard, pudding, or other semiliquid food and beverage products.

### II. Functional Ingredients—Sleep-Inducing Composition

In addition to the stabilizer composition described in Section I, above, the sleep-inducing and/or relaxation beverage composition further includes a novel and unique combination of functional ingredients to create a robust formulation that aids in good quality of sleep, in addition to great taste and improved shelf-life stability. The sleep-inducing composition is used in combination with the stabilizer composition to create a unique, novel, and natural food solution for sleep.

The sleep-inducing composition of the present disclosure includes a unique combination of casein hydrolysate, Zinc, Magnesium, Calcium, Vitamin A, Vitamin K, Vitamin E, Vitamin D, Vitamin B6, Vitamin C, Folate, and Choline. Additionally, an amount of Potassium and Phosphorous can be included with the sleep-inducing composition.

In some embodiments, the casein hydrolysate contains a decapeptide. In additional embodiments the casein hydrolysate is Lactium. The amount of Lactium or casein hydrolysate containing a decapeptide in the sleep-inducing composition is 25 mg to 1500 mg. In some embodiments the amount of lactium is 50 mg, 75 mg, 100 mg, 125 mg, 150 mg, 175 mg, 200 mg 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1,000 mg, 1,025 mg, 1050 mg, 1075 mg, 1,100 mg, 1,125 mg, 1,150 mg, 1,175 mg, 1,200 mg 1,225 mg, 1,250 mg, 1,275 mg, 1,300 mg, 1,325 mg, 1,350 mg, 1,375 mg, 1,400 mg, 1,425 mg, 1,450 mg, 1,475 mg, or 1,500 mg.

In some embodiments, the amount of magnesium in the sleep-inducing composition is 5% to more than 100% of the consumers RDA (recommend daily allowance). In some embodiments that amount of magnesium is 0.5 mg to 350 mg.

In some embodiments, the amount of zinc in the sleep-inducing composition is 5% to more than 100% of the consumers RDA. In some embodiments that amount of zinc is 0.5 mg to 40 mg.

In some embodiments, the amount of calcium in the sleep-inducing composition is 5% to more than 100% of the consumers RDA. In some embodiments that amount of calcium is 125 mg to 2500 mg.

In some embodiments, the amount of vitamin A in the sleep-inducing composition is 5% to more than 100% of the consumers RDA. In some embodiments that amount of vitamin A is 45 mcg to 3000 mcg.

In some embodiments, the amount of vitamin K in the sleep-inducing composition is 5% to more than 100% of the consumers RDA. In some embodiments that amount of vitamin K is 7 mcg to 138 mcg.

In some embodiments, the amount of vitamin E in the sleep-inducing composition is 5% to more than 100% of the consumers RDA. In some embodiments the amount of vitamin E is 0.75 mg to 100 mg.

In some embodiments, the amount of vitamin C in the sleep-inducing composition is 5% to more than 100% of the consumers RDA. In some embodiments the amount of vitamin C is 5 mg to 2000 mg.

In some embodiments, the amount of vitamin D in the sleep-inducing composition is 5% to more than 100% of the consumers RDA. In some embodiments that amount of vitamin D is 0.55 mcg to 100 mcg.

In some embodiments, the amount of vitamin B6 in the sleep-inducing composition is 5% to more than 100% of the consumers RDA. In some embodiments that amount of vitamin B6 is 0.07 mg to 100 mg.

In some embodiments, the amount of folate in the sleep-inducing composition is 5% to more than 100% of the consumers RDA. In some embodiments that amount of folate is 20 mcg to 1000 mcg.

In some embodiments, the amount of choline in the sleep-inducing composition is 1% to more than 100% of the consumers RDA. In some embodiments that amount of choline is 5.5 mg to 3500 mg.

Additionally, the sleep-inducing composition may be used in a variety of products applications including but not limited to 1) baked goods, such as cookies, cakes, pizza crust, tacos, bread, tortillas, bagels, waffles, 2) liquid products including semi-solid puddings, soups, baby foods and smoothies, 3) extruded products such as breakfast cereals, chips, crackers, croutons, ped goods, and 4) other unique products as energy bars, ice creams and Jell-O® or gelatin products.

### III. Protein Shake

Also disclosed herein is a protein shake comprising the stabilizer composition and sleep-inducing composition described in Section I and II above. The protein shake may further include a protein, flavoring agents, coloring agents, micronutrients, preservatives, antifoaming agents, and other ingredients known by those skilled in the art.

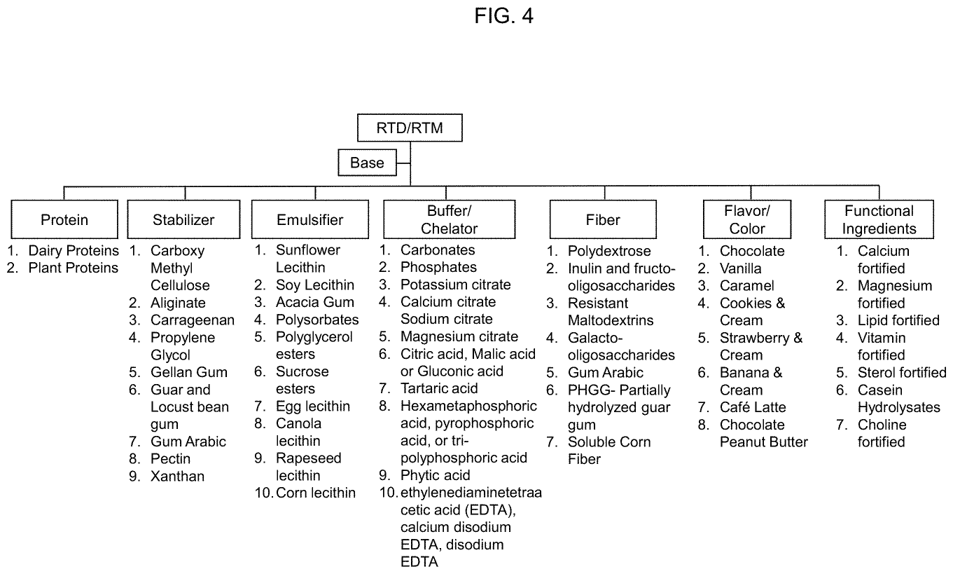

As shown in FIG. 4, the RTD (ready-to-drink) and RTM (ready-to-mix) compositions may include a protein, stabilizer, emulsifier, buffer, fiber, flavoring agents, coloring agents, micronutrients, preservatives, antifoaming agents, and other functional ingredients known to those skilled in the art and may be prepared using the general overview shown in example 1.

The protein shake of the present disclosure could be an RTD (ready-to-drink) or an RTM (ready-to-mix) composition. In some embodiment, the protein may be a dairy protein, a plant protein, or any combination thereof. In some aspects, the protein is a milk protein that may include milk protein concentrate, milk protein hydrolysate, milk protein isolate, whey, or casein. In some additional aspects, the protein may be any plant protein that may include globulins, albumins, glutelins, or prolamins. In some additional aspects the protein may be derived by fermentation processes or via cell cultures.

In some embodiments, the amount of protein in an 8-ounce shake may be from about 5 g to about 35 g for a ready-to-drink (RTD) composition and about 5 g to 20 g of protein for a serving size of about 10 g to 35 g ready-to-mix (RTM)). In some examples, the amount of protein in an 8-ounce shake may be about 5 g, 10 g, 15 g, 20 g, 25 g, 30 g, or about 35 g. In some further examples, the amount of protein in an 8-ounce shake may vary between 5 g to 10 g, about 10 g to about 15 g, about 15 g to about 20 g, about 20 g to about 25 g, about 25 g to about 30 g, or about 30 g to about 35 g. In additional embodiments the amount of protein in a protein shake or in the powder mix to then create the beverage shake end product will be between 1 g and 60 g of protein per serving size, with the serving size of the protein shake being from 5 g to 400 g. Additionally, the amount of protein included in a shake product can be between 0.333 g and about 4.375 g of protein per ounce of a shake product. The desired amount of protein in a the shake product either in the form of an RTD or RTM shake product will depend on the desired amount of protein to provide per serving size for a consumer.

The protein shake of the present disclosure includes at least one food additive. In some embodiments, the food additive may be one or more of a flavoring agent, a coloring agent, a pH adjusting agent, an antifoaming agent, sweeteners, salt, water, micronutrients or functional ingredients, fiber, or any other food additive known to those skilled in the art and any combination thereof.

The protein shake of the present disclosure may include flavoring agents. In some embodiments, the flavoring agents may include natural flavors, artificial flavors, or other flavoring agents known to those skilled in the art and any combination thereof. In some examples, flavoring agents include vanilla flavoring agents, caramel flavoring agents, chocolate flavoring agents, cocoa particles or ingredients, and other flavoring agents known to those skilled in the art. In some aspects, the flavoring agents may have a concentration in the shake varying from 0.05% to 0.5%.

The protein shake of the present disclosure may include an excellent source of fiber. In some embodiments, the fiber source may include inulin, fructo-oligosccharides, resistant maltodextrins, galacto-oligosaccharides, gum arabic, PHGG—Partially hydrolyzed guar gum, soluble corn fiber, or any other fiber sources known to those skilled in the art. In some aspects, the fiber sources may have a concentration ranging from 1.4% to 7.5% to make an excellent source of fiber claim.

The protein shake of the present disclosure may include one or more pH adjusting agents. In some embodiments, the pH adjusting agent may include potassium hydroxide, sodium hydroxide, calcium hydroxide, acetic acid, citric acid or combinations thereof. The pH adjusting agent may be added as needed to achieve a predetermined desired pH.

The protein shake of the present disclosure may include an antifoaming agent. In some embodiments, the antifoaming agent may include insoluble oils, polydimethylsiloxane, or other antifoaming agents known to those skilled in the art. In some aspects, the antifoaming agent may have a concentration of about 0.01%.

The protein shake of the present disclosure may include one or more sweeteners. In some embodiments, the sweetener may be natural or artificial. In some aspects, the sweetener may include cane sugar, sucralose, acesulfame potassium, monk fruit, stevia or other sweeteners known in the art and any combination thereof. In some aspects, the concentration of the sweetener in the protein shake may vary from 0.001% to 0.04%

The protein shake of the present disclosure may include one or more fibers. In some embodiments, the fiber may include soluble corn fiber, galatooligosaccharides, fructo-oligosaccharides etc. In some aspects, the fiber may have a concentration in the protein shake varying from 0.5% to 5%.

The protein shake of the present disclosure may include micronutrients and/or functional ingredients. In some embodiments, the micronutrients may include vitamin A, vitamin E, vitamin D, vitamin K, vitamin B6, folate, zinc, vitamin C, magnesium, calcium, choline, lactium, or any other micronutrient or functional ingredient and any combination thereof.

### VI. Production Method for the Protein Shake

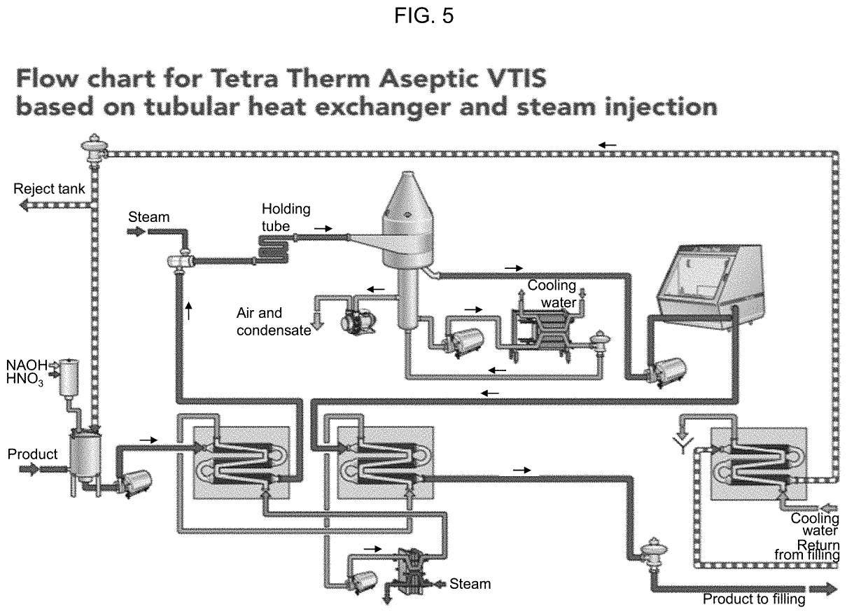

Also, disclosed is a method for producing a protein shake, and in particular a ready-to-drink (RTD) protein shake comprising the stabilizer composition and sleep-inducing composition described in Section I and II above. The protein shake includes numerous other ingredients including but not limited to protein composition, flavoring agents, coloring agents, micronutrients, preservatives, antifoaming agents, and other ingredients known by those skilled in the industry. In one embodiment the RTD shake is prepared based on current industry methods for forming a RTD protein shake. FIG. 5 provides an overview of an example of the typical industry methods for the creation of the RTD protein shake.

The production operation can be divided into four steps: pre-sterilization, production, aseptic intermediate cleaning (AIC) and cleaning in place (CIP) as shown in FIG. 5. The initial step is to prepare the tank or holding/filing machine to add the ingredients for the desired product. The ingredients are then added to the mixing tank and regeneratively pre-heated to about 80° C. (application dependent) in a Tetra Pak® Tubular Heat Exchanger or, as an alternative, in a Tetra Pak® Plate Heat Exchanger. Addition of the ingredients ensures instant heating of the ingredients to sterilization temperature through means of a steam injector by continuous injection of high-pressure steam into the mixture. The mixture then enters a holding tube where it is held at a sterilization temperature for the required time period as defined by production method to ensure proper parameters are met for the production and later packaging of the mixture/product. The mixture then enters the flash vessel where the pressure and temperature drop instantly to aide in product stability and lead up to packaging. The excess water in the form of steam is flashed off and released to not impair the desired product. For optimal product stability, the mixture passes through an aseptic homogenizer before final cooling in the heat exchanger. To aide in continual product production aseptic intermediate cleaning (AIC) can be performed to prolong the production time between full CIPs. When AIC is selected, the mixture creates the product and is displaced by sterile water before cleaning starts. During the AIC sequences, the holding tube is kept at the sterilization temperature, thus keeping the aseptic parts of the unit sterile. After each production run, the unit undergoes cleaning to ensure future batch production is ready.

### Examples

### Example 1: Preparation of the Protein Shake

Protein shakes were prepared based on current industry methods for forming a ready to drink protein shake. FIG. 5 provides an overview of an example of the industry methods for the creation of the protein shake. Following the industry methods a protein shake compositions, such as found in Table 1, were created with the following composition: potassium citrate, carboyl methyl cellulose, high acyl gellan gum, low acyl gellan gum, milk protein concentrate, sunflower lecithin, soluble corn fiber, sunflower oil, cane sugar, sucralose, acesulfame potassium, vitamin A, vitamin E, vitamin D, vitamin K, vitamin B6, folate, zinc, vitamin C, magnesium, calcium, choline, lactium, salt, antifoaming agent, potassium hydroxide and water.

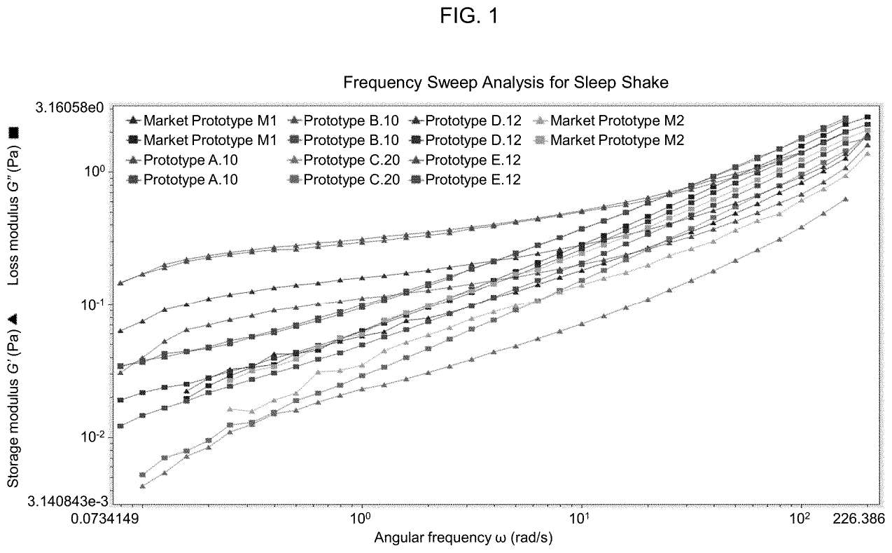

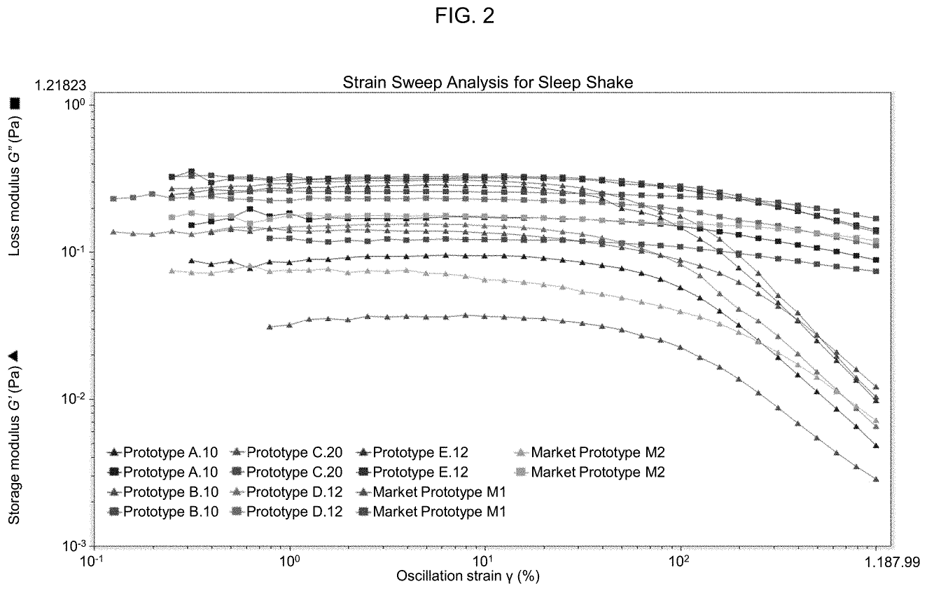

TABLE 1
Ingredient list for the RTD (ready-to-drink) prototypes/examples with great
taste, mouthfeel, texture and robust rheology profiles (details in Figure 1 and Figure 2).

| | Ingredient Statement |
|---|---|
| Prototype | INGREDIENTS: Water, Milk Protein Concentrate, Soluble Corn Fiber, Contains Less |
| A.10 | Than 2% Of Acesulfame Potassium, Calcium Caseinate, Carrageenan, Cellulose Gel |
| | And Cellulose Gum, Choline Chloride, Cocoa Powder (Processed With Alkali), High |
| | Oleic Sunflower Oil, Inulin, Magnesium Phosphate, Maltodextrin, Potassium Hydroxide, |
| | Salt, Antifoam, Sodium Ascorbate (Vit C), Steviol Glycosides, Sucralose, Sunflower |
| | Lecithin, Galacto-Oligosaccharide, Gum, Kelcogel F Gellan Gum, Tripotassium Citrate, |
| | Tripotassium Phospate, Dipotassium Phosphate, Sodium Hexametaphosphate, |
| | Vitamin And Mineral Blend (Dl-Alpha Tocopheryl Acetate [Vitamin E], Zinc Glycinate |
| | Chelate, Ferric Orthophosphate, Vitamin A Palmitate, Niacinamide, Phytonadione |
| | [Vitamin K1], Potassium Iodide, Cholecalciferol [Vitamin D3], Copper Gluconate, |
| | Calcium D-Pantothenate, Manganese Sulfate, Sodium Selenite, Biotin, Sodium |
| | Molybdate, Folic Acid, Thiamine Mononitrate [Vitamin B1], Cyanocobalamin [Vitamin |
| | B12], Pyridoxine Hydrochloride [Vitamin B6], Riboflavin, [Vitamin B2], Chromium |
| | Polynicotinate). |
| Prototype | INGREDIENTS: Water, Milk Protein Concentrate, Soluble Corn Fiber, Contains less |
| B.10 | than 2% of cocoa powder (processed with alkali), High Oleic Sunflower Oil, Cellulose |
| | gel and cellulose gum, Magnesium Phosphate, Salt, Sunflower Lecithin, tripotassium |
| | phospate, dipotassium phosphate, sodium hexametaphosphate, Sodium Ascorbate (Vit |
| | C), Steviol Glycosides, Choline Chloride, |
| | Vitamin and Mineral Blend (DL-alpha tocopheryl acetate [vitamin E], zinc glycinate |
| | chelate, ferric orthophosphate, vitamin A palmitate, niacinamide, phytonadione [vitamin |
| | K1], potassium iodide, cholecalciferol [vitamin D3], copper gluconate, calcium D- |
| | pantothenate, manganese sulfate, sodium selenite, biotin, sodium molybdate, folic acid, |
| | thiamine mononitrate [Vitamin B1], cyanocobalamin [vitamin B12], pyridoxine |
| | hydrochloride [vitamin B6], riboflavin, [vitamin B2], chromium polynicotinate), Potassium |
| | Hydroxide, Gellan Gum, Sucralose, Antifoam, Kelcogel F Gellan Gum, Acesulfame |
| | potassium. |
| Prototype | INGREDIENTS: Water, Milk Protein Concentrate, contains less than 2% of calcium |
| C.20 | caseinate, cocoa powder (processed with alkali), High Oleic Sunflower Oil, Inulin, |
| | Cellulose gel and cellulose gum, Magnesium Phosphate, tripotassium phospate, |
| | dipotassium phosphate, sodium hexametaphosphate, Sunflower Lecithin, Salt, Casein |
| | Hydrolysate, Sucralose, Sodium Ascorbate (Vit C), Choline Chloride. |
| | Vitamin and Mineral Blend (dl-alpha tocopheryl acetate [vitamin E], zinc glycinate |
| | chelate, pyridoxine hydrochloride [vitamin B6], vitamin A palmitate, folic acid, |
| | phytonadione [Vitamin K1], cholecalciferol [vitamin D3]), Potassium Hydroxide, HM Pure |
| | Gellan Gum, Acesulfame potassium, Antifoam, Kelcogel F Gellan Gum, Carrageenan. |
| Prototype | INGREDIENTS: Water, Milk Protein Concentrate, Contains less than 2% of MFGM, |
| D.12 | cocoa powder (processed with alkali), GOS Galacto-Oligosaccharide, Cellulose gel and |
| | cellulose gum, Magnesium Phosphate, Salt, Tripotassium Citrate, Casein Hydrolysate, |
| | Sucralose, Sunflower Lecithin, Sodium Ascorbate (Vit C), Choline Chloride |
| | Vitamin and Mineral Blend (dl-alpha tocopheryl acetate [vitamin E], zinc glycinate |
| | chelate, pyridoxine hydrochloride [vitamin B6], vitamin A palmitate, folic acid, |
| | phytonadione [Vitamin K1], cholecalciferol [vitamin D3]), Potassium Hydroxide, HM Pure |
| | Gellan Gum, Acesulfame potassium, Antifoam, Gum, Kelcogel F Gellan Gum |
| Prototype | INGREDIENTS: Water, Milk Protein Concentrate, Contains less than 2% of MFGM, |
| E.12 | cocoa powder (processed with alkali), Test, GOS Galacto-Oligosaccharide, Cellulose |
| | gel and cellulose gum, Magnesium Phosphate, Tripotassium Citrate, Salt, Sugar, |
| | Casein Hydrolysate, Sucralose, Sunflower Lecithin, Sodium Ascorbate (Vit C), Choline |
| | Chloride, |
| | Vitamin and Mineral Blend (dl-alpha tocopheryl acetate [vitamin E], zinc glycinate |
| | chelate, pyridoxine hydrochloride [vitamin B6], vitamin A palmitate, folic acid, |
| | phytonadione [Vitamin K1], cholecalciferol [vitamin D3]), Potassium Hydroxide, HM Pure |
| | Gellan Gum, Acesulfame potassium, Antifoam, Kelcogel F Gellan Gum |

### Example 2: Protein Powder Production for RTM Shakes

Powder manufacturing steps include pre-weighing the raw materials, followed by screening and loading them into the blender. After loading is complete, the raw materials are typically blended for 5-25 mines (depending on the powder type). Once blending is complete, the blends are discharged into super sacks and packaged into containers.

Mixing Instructions: Protein powers typically clump when missed with hot water (greater than 78 degrees F.). To overcome this challenge, several mixing options were explored, and it was identified that mixing protein powder in cold/room temperature water 36-70 degrees F. and microwaving/heating the liquid provides the best consumer experience in terms of texture (without clumps). Ideal mixing instruction include: stirring the sleep protein powder with water (room temperature/cold water), and microwaving for 1 minute (or until hot). The powder mixes entirely in cold water and stays in suspension for at least 20-30 minutes when warmed up, depending on the temperature to which it is heated.

Powder Prototypes in Table 2 were prepared using the processes detailed above. Table 2 includes the ingredient list for the RTM examples with improved taste, mouthfeel and texture.

TABLE 2
Ingredient list for the RTM (ready-to-mix) prototypes/examples with great taste,
mouthfeel, and texture.

| | Ingredient Statement |
|---|---|
| R1 | INGREDIENTS: Milk Protein Isolate, Cocoa Powder (Processed with Alkali), |
| | Magnesium Phosphate, Salt, contains less than 2% of the following: Casein |
| | Hydrolysate, Milk Minerals, Vitamin and Mineral Blend (Dl-Alpha Tocopheryl Acetate |
| | [Vitamin E], Zinc Glycinate Chelate, Pyridoxine Hydrochloride [Vitamin B6], Vitamin A |
| | Palmitate, Folic Acid, Phytonadione [Vitamin K1], Cholecalciferol [Vitamin D3]), |
| | Sucralose, Natural and Artificial Flavor, Sodium Ascorbate |
| R2 | INGREDIENTS: Milk Protein Isolate, Cocoa Powder (Processed with Alkali), |
| | Magnesium Phosphate, Salt, contains less than 2% of the following: Casein |
| | Hydrolysate, Milk Minerals, Vitamin and Mineral Blend (Dl-Alpha Tocopheryl Acetate |
| | [Vitamin E], Zinc Glycinate Chelate, Pyridoxine Hydrochloride [Vitamin B6], Vitamin A |
| | Palmitate, Folic Acid, Phytonadione [Vitamin K1], Cholecalciferol [Vitamin D3]), |
| | Sucralose, Natural and Artificial Flavor, Sodium Ascorbate |
| R3 | INGREDIENTS: Milk Protein Isolate, Cocoa Powder (Processed with Alkali), |
| | Magnesium Phosphate, Salt, contains less than 2% of the following: Casein |
| | Hydrolysate, Milk Minerals, Vitamin and Mineral Blend (Dl-Alpha Tocopheryl Acetate |
| | [Vitamin E], Zinc Glycinate Chelate, Pyridoxine Hydrochloride [Vitamin B6], Vitamin A |
| | Palmitate, Folic Acid, Phytonadione [Vitamin K1], Cholecalciferol [Vitamin D3]), |
| | Sucralose, Natural and Artificial Flavor, Sodium Ascorbate |
| R4 | INGREDIENTS: Milk Protein Isolate, Cocoa Powder (Processed with Alkali), |
| | Magnesium Phosphate, Salt, contains less than 2% of the following: Casein |
| | Hydrolysate, Milk Minerals, Vitamin and Mineral Blend (Dl-Alpha Tocopheryl Acetate |
| | [Vitamin E], Zinc Glycinate Chelate, Pyridoxine Hydrochloride [Vitamin B6], Vitamin A |
| | Palmitate, Folic Acid, Phytonadione [Vitamin K1], Cholecalciferol [Vitamin D3]), |
| | Sucralose, Natural and Artificial Flavor, Sodium Ascorbate |
| R6 | INGREDIENTS: Milk Protein Isolate, Cocoa Powder (Processed with Alkali), |
| | Magnesium Phosphate, Salt, contains less than 2% of the following: Casein |
| | Hydrolysate, Milk Minerals, Vitamin and Mineral Blend (Dl-Alpha Tocopheryl Acetate |
| | [Vitamin E], Zinc Glycinate Chelate, Pyridoxine Hydrochloride [Vitamin B6], Vitamin A |
| | Palmitate, Folic Acid, Phytonadione [Vitamin K1], Cholecalciferol [Vitamin D3]), |
| | Sucralose, Natural and Artificial Flavor, Sodium Ascorbate |
| R8 | INGREDIENTS: Milk Protein Isolate, Cocoa Powder (Processed with Alkali), |
| | Magnesium Phosphate, Salt, contains less than 2% of the following: Casein |
| | Hydrolysate, Milk Minerals, Vitamin and Mineral Blend (Dl-Alpha Tocopheryl Acetate |
| | [Vitamin E], Zinc Glycinate Chelate, Pyridoxine Hydrochloride [Vitamin B6], Vitamin A |
| | Palmitate, Folic Acid, Phytonadione [Vitamin K1], Cholecalciferol [Vitamin D3]), |
| | Sucralose, Natural and Artificial Flavor, Sodium Ascorbate |

### Example 3: Rheological Overview

For the example protein shake compositions, the rheology overview is provided (Please see below the Strain Sweep and Frequency Sweep analysis). See FIG. 1 and FIG. 2. The frequency sweep and strain sweep below indicate that the magnitude loss and storage modulae of prototypes D.12 and E12 are higher than the market prototypes M1 and M2. Storage modulus (G′) indicates the viscous properties of the product and loss modulus (G′) indicates the elastic properties. As noticed below in the strain sweeps, G″>G′ across majority of the prototypes indicate more stability. Also, upon close comparison, it is evident that the difference of magnitude between G″ and G′ of prototypes D.12 and E.12 is higher than that between the market prototypes M1 and M2, indicates higher stability in the former than the latter. Furthermore, in addition to the magnitude of G″ and G′, another critical metric of stability are critical strain value and the linear viscoelastic region (LVR), which is indicated by the plateau region of loss modulus (G′) in the strain sweeps below. Typically, longer horizontal span of LVR indicates higher stability and stable network structure, as observed below when for example, E.12 is compared to M2. And lastly, cross-over points are critical in determination of overall product stability and early detection of physical defects. As observed in the strain sweeps below, there are no cross-over observed in any of the prototypes tested, indicating a robust and stable product.

### Example 4: Sensory Testing

Sensory testing was conducted on the novel protein shake compositions from Example 1 and compared to current market products. The below chart provides the shake examples used for the sensory testing. Each of these shakes were based on compositions produced under Example 1 above. The sensory testing focused on the sensory taste experiences, as outlined in the parameters below, that include mouthfeel and flavor and the consumers response to the shake compositions.

Table 3: Ingredient list for the RTD shake examples 1 and 2 used in the sensory testing.

TABLE 3
Ingredient list for the RTD shake examples 1 and 2 used in the sensory testing.

| | | Ingredient Statement |
|---|---|---|
| Shake | Derivative | INGREDIENTS: Water, Milk Protein Concentrate, Contains Less Than 2% |
| example 1 | of D.12 | Of Acesulfame Potassium, Casein Hydrolysate, Cellulose Gel And |
| (solid) | and E.12 | Cellulose Gum, Choline Chloride, Cocoa Powder (Processed With Alkali), |
| | above | Gellan Gum, High Oleic Sunflower Oil, MFGM, Magnesium Phosphate, Milk |
| | | Minerals, Potassium Hydroxide, Salt, Antifoam, Sodium Ascorbate (Vit C), |
| | | Soluble Corn Fiber, Sucralose, Sugar, Sunflower Lecithin, Galacto- |
| | | Oligosaccharide, Kelcogel F Gellan Gum, Tripotassium Citrate, |
| | | Vitamin And Mineral Blend (DI-Alpha Tocopheryl Acetate [Vitamin E], Zinc |
| | | Glycinate Chelate, Pyridoxine Hydrochloride [Vitamin B6], Vitamin A |
| | | Palmitate, Folic Acid, Phytonadione [Vitamin K1], Cholecalciferol [Vitamin |
| | | D3]). |
| Shake | Derivative | INGREDIENTS: Water, Milk Protein Concentrate, Soluble Corn Fiber, |
| example 2 | of C.20 | Contains Less Than 2% Of Acesulfame Potassium, Casein Hydrolysate, |
| (square | above | Cellulose Gel And Cellulose Gum, Choline Chloride, Cocoa Powder |
| pattern) | | (Processed With Alkali), High Oleic Sunflower Oil, Magnesium Phosphate, |
| | | Natural And Artificial Flavors, Salt, Antifoam, Sodium Ascorbate (Vit C), |
| | | Sucralose, Sugar, Sunflower Lecithin, HM Pure, Kelcogel F Gellan Gum, |
| | | Tripotassium Citrate, |
| | | Vitamin And Mineral Blend (DI-Alpha Tocopheryl Acetate [Vitamin E], Zinc |
| | | Glycinate Chelate, Pyridoxine Hydrochloride [Vitamin B6], Vitamin A |
| | | Palmitate, Folic Acid, Phytonadione [Vitamin K1], Cholecalciferol [Vitamin |
| | | D3]). |

### Sensory Testing Parameters:

Overall liking data were collected by asking consumers to describe their overall opinion of the product using a 9-pt liking scale
Overall flavor liking data were collected by asking consumers to describe their overall opinion of the product's flavor using a 9-pt liking scale
Overall texture liking data were collected by asking consumers to describe their overall opinion of product's mouthfeel using a 9-pt liking scale

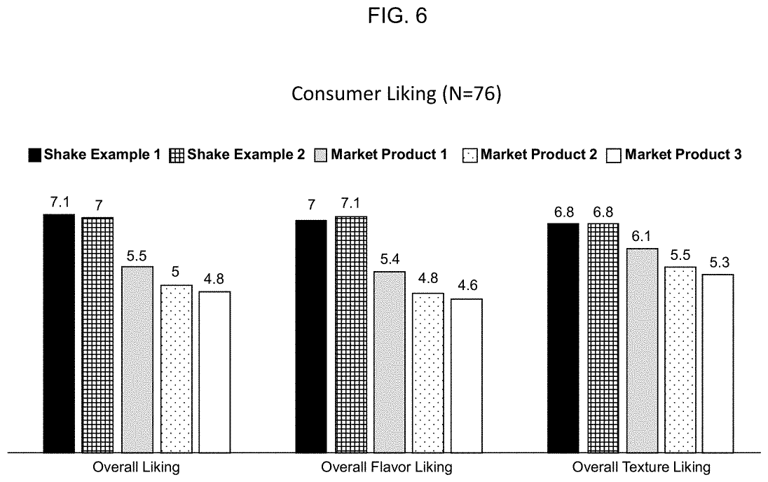

The sensory testing data (FIG. 6) demonstrates the RTD sleep-inducing protein shake has an improved overall liking, overall flavor and overall texture as compared to current RTD protein shakes found on the market. These characteristics are desired by a consumer and demonstrate the unique beverage composition.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

What is claimed is:

1. A sleep-inducing protein shake composition comprising:
a protein;
a stabilizer composition, wherein the stabilizer composition comprises:
at least one stabilizing agent selected from the group consisting of a cellulose gel/gum, a starch, a gellan gum, and any combination thereof;
a chelator; and
an emulsifier; and
a sleep-inducing composition, wherein the sleep-inducing composition includes casein hydrolysate, zinc, calcium, magnesium, vitamin A, vitamin K, vitamin E, vitamin D, vitamin B6, vitamin C, folate, choline and combinations thereof; and
at least one food additive.

2. The composition of claim 1, wherein the cellulose gel/gum is selected from the group consisting of carboxymethyl cellulose, microcrystalline cellulose, and any combination thereof and wherein the cellulose gel/gum has a concentration of about 0.01% to about 1.25% of the sleep-inducing protein shake.

3. The composition of claim 1, wherein the gellan gum is selected from the group consisting of high acyl gellan gum, low acyl gellan gum, and any combination thereof and wherein the high acyl gellan gum has a concentration of about 25 ppm to about 600 ppm and wherein the low acyl gellan gum has a concentration of about 25 ppm to about 250 ppm.

4. The composition of claim 3, wherein the ratio of high acyl gellan gum to low acyl gellan gum is between about 1:1 to about 4:1.

5. The composition of either of claim 1, further includes a starch wherein the starch has a concentration of 0.3% to about 3% of the sleep-inducing protein shake.

6. The composition of claim 1, wherein the chelator is selected from the group consisting of potassium citrate, calcium citrate, sodium citrate, magnesium citrate, and any combination thereof and wherein the chelator has a concentration of about 0.01% to about 0.6% of the sleep-inducing protein shake.

7. The composition of claim 1, wherein the protein is selected from the group consisting of dairy proteins, plant proteins, and any combination thereof.

8. The composition of claim 7, wherein the dairy protein is selected from the group consisting of milk protein concentrate, milk protein hydrolysate, milk protein isolate, whey protein, and any combination thereof.

9. The composition of claim 7 wherein the plant protein is selected from the group consisting of soy protein, pea protein, hydrolyzed plant proteins or any combinations thereof.

10. The composition of claim 1, wherein the fiber has a concentration of about 0.7 g per 1 ounce.

11. The composition of claim 1, wherein the emulsifier is a lecithin, polyglycerol esters, sucrose esters, acacia gum or any combinations thereof and wherein the emulsifier has a concentration of about 0.03% to about 0.8%.

12. The composition of claim 11, wherein the lecithin is sunflower lecithin or soy lecithin.

13. The composition of claim 1, further including phosphorus.

14. The composition of claim 1, wherein the composition includes at least one additional ingredient selected from the group consisting of an oil (medium-chain triglyceride (MCT) oil, canola oil, corn oil, soy oil, high oleic sunflower oil (HOSO), pumpkin seed oil, grapeseed oil, peanut oil, sesame oil, flaxseed oil, sunflower seed oil, vegetable oil, or any combinations thereof), an oligosaccharide, a galactooligosaccharide, milk fat globule membrane, a fiber source and any combination thereof.

15. The composition of claim 1, wherein the sleep-inducing wherein the casein hydrolysate contains a decapeptide, or wherein the lactium is used in place of the casein hydrolysate.

16. The composition of claim 1, wherein the sleep-inducing protein shake composition is a ready to drink shake composition.

17. A sleep inducing ready to mix protein shake powder composition comprising:
protein;
a stabilizer composition, wherein the stabilizer composition comprises:
at least one stabilizing agent selected from the group consisting of a cellulose gel/gum, a starch, a gellan gum, and any combination thereof;
a chelator; and
an emulsifier; and
a sleep-inducing composition, wherein the sleep-inducing composition includes casein hydrolysate, zinc, calcium, magnesium, vitamin A, vitamin K, vitamin E, vitamin D, vitamin B6, vitamin C, folate, choline and combinations thereof
at least one food additive.

18. A stabilizer composition for use in a food or beverage composition comprising:
at least one stabilizing agent selected from the group consisting of a cellulose gel/gum, a starch, a gellan gum, and any combination thereof;
a chelator; and
an emulsifier.

19. A sleep-inducing protein shake composition comprising:
a protein;
a stabilizer composition, wherein the stabilizer composition comprises:
at least one stabilizing agent selected from the group consisting of a cellulose gel/gum, a starch, a gellan gum, and any combination thereof;
a chelator; and
an emulsifier; and
a sleep-inducing composition, wherein the sleep-inducing composition includes a casein hydrolysate containing a decapeptide in an amount of 25 mg to about 1500 mg, zinc in an amount of 0.5 mg to about 40 mg, calcium in an amount of 125 mg to 2500 mg, magnesium in an amount of 0.5 mg to 350 mg, vitamin A in an amount of 45 mcg to 3000 msg, vitamin K in an amount of 7 mcg to about 138 mcg, vitamin E in an amount of 0.75 mg to 100 mg, vitamin D in an amount of 0.55 mcg to 100 mcg, vitamin B6 in an amount of 0.07 mg to 100 mg, vitamin C in an amount of 5 mg to 2000 mg, folate in an amount of 20 mcg to 1000 mcg, choline in an amount of 5.5 mg to 3500 mg and combinations thereof; and
at least food additive.

20. The sleep-inducing protein shake of claim 19, wherein the sleep-inducing composition further includes potassium and/or phosphorous.

21. The sleep-inducing protein shake of claim 19, wherein the casein hydrolysate containing a decapeptide is Lactium.
